(19) United States
(12) Patent Application Publication
(10) Pub. No.: US 2005/0196410 A1
(43) Pub. Date: Sep. 8, 2005

(54) Seaweed extract composition for treatment of inflammation

(76) Inventor: Bruce Alan Daniels, Oklahoma City, OK (US)

Correspondence Address: Ali Kamarei, Esq., 280 Colorado Ave., Palo Alto, CA, 94301, US

(21) Appl. No.: 10/794,892

(22) Filed: Mar. 5, 2004

(51) Int. Cl. A61K 35/80; A61K 31/715

(52) U.S. Cl. 424/195.17; 514/54

(57) ABSTRACT

The invention of comprises of a method and composition for retardation of Inflammation in a mammal comprising the step of administering to a mammal an amount of seaweed extract in effective doses. The invention also comprises a composition and method for preserving a healthy inflammatory response in a mammal comprising the step of administering to a mammal an amount of seaweed extract in effective doses. The seaweed is selected from the group consisting of brown algae, red algae and green algae, wherein the seaweed extract contains a polysaccharide. Optionally, the seaweed extract contains between approximately 63 mole % to 78 mole % Rhamnose. Optionally, the seaweed extract contains between approximately 6.5 mole % to 9.2 mole % Xylose. Optionally, the composition of Rhamnose and Xylose are present in the seaweed extract are in amounts between a ratio of approximately 12 Rhamnose to 1 Xylose and approximately 8 Rhamnose to 1 Xylose.

### FIELD

This invention relates to a pharmacological composition and method that provides for treatment of pathologic inflammation as well as preserving a an effective physiologic inflammatory response. This composition is preferably used for patients susceptible to or suffering from increased C-Reactive Protein, or elevated levels of inflammation. The composition and method of the invention therefore, treats various diseases related to inflammation including, atherosclerosis, arthritis, colitis, hepatitis, glial cell and brain cell inflammation.

### BACKGROUND

Inflammation and inflammatory disorders and diseases and their associated complications are a principal cause of cellular structure and function changes. For example, with regard to only arthritis, it is the leading cause of disability among U.S. adults. It limits everyday activities for more than 7 million Americans. By 2020, an estimated 12 million Americans will be limited in daily activities because of arthritis. Arthritis is not just an old person's disease: nearly two-thirds of people with arthritis are younger than 65 years.

Existing treatments currently only treat the inflammatory processes at the level of a specific mediator, as in the case of Prostaglandin inhibitors, such as NSIAD drugs and ibuprofen and Cox2 inhibitors, such as Celebrex®.

These treatments have serious shortcomings as they only treat a single mediator out of several which are simultaneously operative in the processes at the cell and cell matrix level. Thus leaving others still active and unopposed.

The present invention differs in that it works to prevent the pro-inflammatory state by presenting or retarding the dysfunctional transcription of DNA sequences, which lead to the inflammatory state. In fact, similar transcriptional processes are involved in a host of inflammatory diseases, thus the current invention has numerous disease applications.

None of these treatment methods are directed towards the underlying disease processes, the molecular causes of the disease or disorders, or towards restoring the structure and function of the endothelium, which is a principal line of defense against inflammation in cells, tissues, and cell matrixes.

In view of the foregoing, there is a significant need for a pharmacological composition and method that is directed towards treating the underlying inflammation disease process, and towards restoring the structure and improving the functions of the endothelium and the extracellular gel or matrix.

It is an objective of the present invention to provide a treatment, which is directed to preventing and minimizing dysfunctional atomic and molecular interactions within the human cellular matrix or cellular environment, which lead to inflammation.

It is another objective of the present invention to provide a treatment that is directed to retarding adverse consequences of free radicals generated in human cellular matrix. It is also another objective of the present invention to stimulate an increased production of nitric oxide within human cellular matrix or cellular environment.

It is yet another objective of the present invention to treat inflammation, in particular, atherosclerosis, arthritis, colitis, hepatitis, glial cell and brain cell inflammation, without appreciably increasing blood anticoagulation activity in patients.

It is also another objective of the present invention to reduce C-Reactive Protein levels in the plasma. CRP leads to NFKB activation and transcription of a set of inflammatory genomic transcriptional processes which are reduced by use of the present invention.

Another objective of the present invention is to retard and prevent inflammatory cells from binding to and traversing thought the endothelium. By decreasing the number of such inflammatory cells in tissues, inflammation is reduced.

### SUMMARY

The invention of comprises of a method and composition for retardation of Inflammation in a mammal comprising the step of administering to a mammal an amount of seaweed extract in effective doses.

The invention also comprises a composition and method for preserving a healthy inflammatory response in a mammal comprising the step of administering to a mammal an amount of seaweed extract in effective doses.

The seaweed is selected from the group consisting of brown algae, red algae and green algae, wherein the seaweed extract contains a polysaccharide. Optionally, the polysaccharide is Rhamnose, in approximately 63-78 mole %. The seaweed extract optionally also contains approximately 6.5-9.2 mole % Xylose. Optionally, Rhamnose and Xylose are present in the seaweed extract in amounts in a ratio of approximately 12 Rhamnose to 1 Xylose and 8 Rhamnose to 1 Xylose.

The composition and method of the invention further comprising the co-administration of a nitrogen donor or nitric oxide donor together with the seaweed extract, wherein the nitrogen donor is selected from the group consisting of L-Arginine and Lysine.

An advantage of the method and composition of the invention is that it possesses extremely potent anti-inflammatory activity and other inhibitory effects on cell surface adhesion of monocytes to the endothelium surface.

Another advantage of the described composition is that there is less peptide residual in extracting the composition from plant cells as compared to heparin from animal cells. Hence, it is less allergic reaction prone and has fewer immunogenic properties.

Yet another advantage is that since seaweed extract is from plant cells, it has no potential for the transmission of potentially lethal and serious prion diseases such as mad cow disease.

Another advantage is that seaweed extract has no potential for activating Platelet Factor IV and resulting in immune complex destruction of platelets as seen with heparin administration.

It is also another advantage of the present invention in that it reduces C-Reactive Protein levels in the plasma.

### BRIEF DESCRIPTION OF DRAWINGS

FIG. 1 is a chemical representation of Rhamnan Sulphate and Rhamnan Sulfate salt of Arginine.

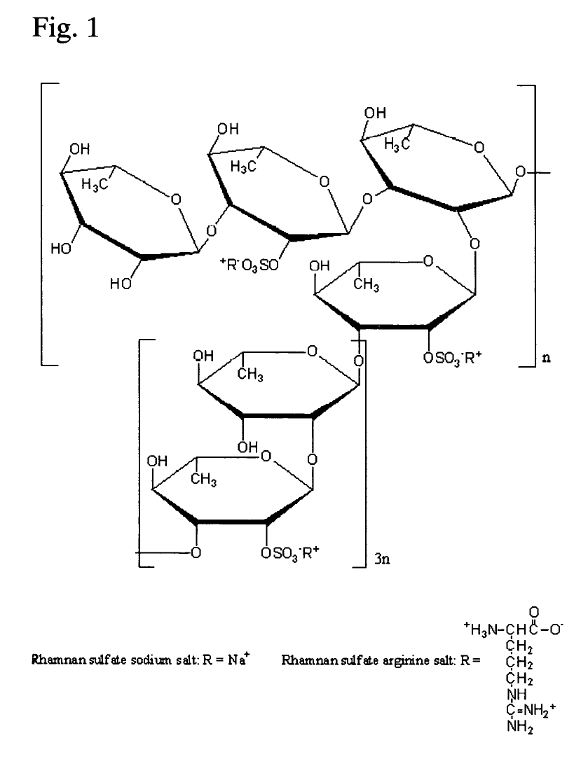

### DETAILED DESCRIPTION

The inventor, in U.S. Pat. Nos. 6,255,296 and 6,495,530, outlines the fact that a cellular environment (cellular matrix or gel matrix) composed of charged polymers-highly charged peptide-water polymers, such as heparin-arginine-water is responsible for controlling the structure and ultimately the function of human cells within this cellular environment. As the human blood vessel endothelium is only one cell thick, it too operates within this charged polymer-highly charged peptide-water environment. Thus, this charged polymer-arginine-water environment impacts such important functions of the cells by effecting protein distribution and functionality, cell signaling processes, genetic or DNA-RNA transcription regulation, and the physical/chemical properties of cells, including blood vessel wall cells. It should also be noted that heparins or heparin domains within these polymer structures are members of the group commonly referred to as endogenous heparans. Exogenous heparans, including heparin, have functions, which protect the endogenous heparans.

The present invention is directed to a formulation for treatment of the gel matrix or cellular environment and retardation of inflammation and endothelial dysfunction. Retardation is defined as to slow down the progression or development of inflammation, or to reduce the risk of diseases associated with inflammation. Furthermore, as is evident from clinical experiments described below, the present formulation is also effective in reducing the risk of development of inflammation in healthy individuals. Thus, the present composition and method is also effective in preserving and promoting a healthy inflammatory response in the body.

In accordance with the invention, a patient susceptible to or suffering or affected by inflammation. Such inflammatory diseases include atherosclerosis, arthritis, colitis, hepatitis, glial cell and brain cell inflammation. Additionally, a healthy individual, who over time would likely develop inflammation or suffer from chronic low level inflammation, is also treated with a dose or amount of the composition of the present invention.

The definition of the term extract used in seaweed extract is not limited to the extraction method in Example 1, rather the term is used broadly to include crushing the seaweed and mixing with water or other ingredients; chopping, grinding, mincing, or forming a paste of the seaweed, processing the seaweed into a dry powder, extruding, fermenting, or any other process by which the polysaccharide, such as Rhamnose or other polysaccharides remain in the extract.

The seaweed is preferably selected from the group consisting of brown algae, red algae and green algae. Brown algae are selected from the group consisting of Fucus vesiculosus, Fucus Evanescens, Laminaria brasiliensis, or Ascophylum nodosum. Soliera robusta is an example of red algae. Green algae is selected from the group consisting of Monostroma nitidium, Monostroma zosteticola, Monostroma angicava, Monostroma lattissimum, Monostroma pulchrum, Monostroma fusem, Monostroma grevillei, Entoromorpha compressa, Ulva arasakii, Ulva Pertussa, Cladophora denna, Cladophora rugulosa, Chaecomorpha spiralis, Chaecomorpha crassa, Spongomorpha duriuscula, Codium fragile, Codium divaricaium Codium latum, or Caulerpa okamarai.

An amount of seaweed extract containing Rhamnose or other polysaccharide, such that it is sulfinated either synthetically or by natural metabolism in the mammal, and which is useful and effective in retardation of cardiovascular disorder, is defined primarily by clinical response in a patient equivalent to about 2,000 IU to 200,000 IU heparin activity. A more preferred range of an effective amount of seaweed extract is equivalent to about 5,000 to 20,000 IU heparin activity. A most preferred range of an effective amount of seaweed extract is equivalent to between about 8,000 IU and 12,000 IU heparin activity.

Sulfination of a polysaccharide is defined as attaching a sulfate group to the polysaccharide to form a sulfated moiety of the polysaccharide. For example, forming Rhamnan Sulfate from Rhamnose, or Xylose Sulfate from xylose. Rhamnan Sulfate backbone can be substituted at the 2-, 3- or 4- positions with minor amounts (less than 10% in total) of galacturoinc acid, xylose and arabinose.

When absorbed into the charged polymer-highly charged peptide-water matrix, the polysaccharide in the seaweed extract protects and reinforces structure and roles of endogenous heparans. Whatever the mechanism, the polysaccharide in the seaweed extract has a potent effect on surface anti-inflammatory effects on the endothelium rather than the plasma anticoagulation.

Localization of administered heparin or heparin analogues to cell surfaces (e.g. endothelial surfaces) by oral administration inhibits thrombotic activity within and on artery and blood vessel surfaces without the inhibition of plasma clotting factors seen with currently available anticoagulants.

The polysaccharide in the seaweed extract is characterized such that it should be administered in an amount sufficient to exert anti-inflammatory effects on the endothelial cells, while not increasing the patient's risk of internal or external hemorrhaging and effectively maintaining integrity and functionality of the cellular membranes and surrounding environments of the endothelial cells.

Effective doses of the seaweed extract vary with the particular individual's condition and the method of administration. For example, it is noticed that subcutaneous injection of heparin results in greater concentration in the cellular and membrane domains than intravenous injection, and it is the inventor's observation that oral heparan sulphates localizes almost exclusively to cell surface membranes, especially the endothelium. Thus, the preferred method of administration of the seaweed extract for the present invention is through the oral route, while the least preferred method is via intravenous injection.

The compound of the present invention is optionally formulated for oral, sublingual, subcutaneous, intravenous, transdermal or rectal administrations in dosages and in admixture with pharmaceutical excipients or vehicles including implantation or controlled-release devices. For example, the compound of the seaweed extract is optionally dispersed in a physiologically acceptable, non-toxic liquid vehicle, such as water.

Alternatively, the compound can be given in tablet, capsule, powder, granules, coated tablet form, or mixed with various food stuffs such as; cereals, bread, drinks, health bars, juices, concentrates, canned food, ice cream, water, staple goods such as wheat, corn, barley, and oat in any form, processed or not, or taste maskers such as sugar or ascorbic acid, or other functional foods. The compound is made using conventional methods, and may be mixed with conventional pharmaceutical auxiliaries, such as binders, fillers, preservatives, tablet disintegrators, flow regulators, plasticizers, wetting agents, dispersants, emulsifiers, solvents, retarding agents and/or anti-oxidants. It is also optionally contained or formed into a complex with lipids in various formulations and molecular arrangements.

An efficiently operating homeostatic system is crucial to cellular function within mammalian organisms. In a healthy state, there is formed a gel matrix of heparin, highly charged peptide and water polymers, which houses a plurality of other molecules by accommodating dynamic binding of and release of such molecules without reaching concentration levels which destroy the gel structure and its regulatory functionalities.

Long chain charged polymer strands are an organizing determinant for membranes, proteins, receptors, ion channels, cell organelles, nuclear membranes, membrane pores, and other complex cellular constituents. The polymers and highly charged amino acids such as Arginine organize water into arenas for confining bilipid layer membranes, for example, creating cell turgor and form and limiting hydrolytic properties of water on other molecular structures.

A healthy gel matrix is formed from endogenous charged polymers, endogenous arginine and water. An unhealthy state of a gel matrix has some of the highly charged peptides molecules cleaved out of the gel. Likewise, charged polymers have been removed from the gel. There are thus created gaps between charged polymers into which other molecules can embed or pass through.

The healthy gel structure has a conformation that preferentially supports interaction and binding of foreign molecules. The capacity to accommodate intrusions of such molecules before the gel structure collapses and loses its functionality is an important characteristic of the gel system.

The permeability of the membranes thus allows macromolecules or cells to enter and traverse the gel. For example, cholesterol, clotting factors and water traverse the gel reaching a bilipid layer, or other subendothelial locations. In addition, ionic strength, flow stress, heat, osmotic pressure or other forms of energy transfer to the gel can deteriorate the properties of the gel as described above.

These intrusions result in a displacement of arginine and decreased generation of nitric oxide as an additional effect. Intrusions limit the binding capacity of the heparin for arginine and other molecules within the gel.

In order to reverse this disruption of the gel matrix caused by the removal of arginine and/or heparin, the present invention employs the polysaccharides in seaweed extract (algopolysaccharides) to maintain and rejuvenate the gel matrix and its functionality. These algopolysaccharides are selected from the group consisting of; Rhamnose, Xylose, Galactose, and Mannose. In this regard, the present invention utilizes the polysaccharides in seaweed extract, which is particularly high in Rhamnose, to give optimal pore closure and stabilization, and number and distribution of binding sites, wherein signaling, anti-proliferation, and anti-inflammatory effects are maintained. Thus, the homeostasis-promoting functionalities of heparin, arginine, and charged polymer-highly charged peptide-water gel matrix, resultant from the herein-described composition, retard continuous and accumulative change and injury to cellular domains. By this retarding effect inflammation is minimized.

Addition of a sulfated Rhamnose polysaccharide, Rhamnan Sulfate, to the gel system protects the functionality of both heparin and the arginine in the gel matrix. In the extragellular medium, the ability of heparin to bind and quiesce molecules is augmented by simultaneous addition of Rhamnan Sulphate, wherein Rhamnan Sulphate binds to extragellular potentially-intruding molecules, thus allowing existing gellular charged polymers to associate with gellular arginine.

Nitric oxide produced from arginine is an important physiological mediator. The enzyme responsible for nitric oxide production, nitric oxide synthase, requires CA++ and Calmodulin. The functionality of the heparin-arginine gel includes its binding and regulation of CA++ and Calmodulin. By regulating Calmodulin activity, the effects of Rhamnan Sulphate on the charged polymer-arginine gel regulates nitric oxide synthase activity responsible for nitric oxide production.

Arginine acts as a nitrogen donor for the production of Nitric Oxide. However, it is understood that lysine is optionally used instead of Arginine as a nitrogen donor. A larger dose of lysine may be required to be as effective as a smaller dose of arginine. Other nitric oxide donors also may be optionally used.

The binding of water, small anions and cations within the charged polymer-arginine-water gel is facilitated by pi-bonding properties inherent in the saccharide ring structure within the charged polymers. Changes in the shared electron density and electrical charge variation regulated the state of solvation and conformation of the gel polymers. Thus, small anion and cation binding induces changes in the state of salvation, changes in catalytic and hydrolytic properties of water, and changes in capacity of the gel to bind water and other molecules. Low to high molecular weight Rhamnose derived from seaweed extract, preferably having a high degree of sulfation, is preferably used.

It is believed that Rhamnan sulfate on the endothelium reduces attachment of inflammatory cells and their passage into healthy tissues. Heparin binds super oxide dismutase, which absorbs high-energy electrons and deactivates free radicals. The polysaccharides derived from the seaweed extract, including their sulfated moiety such as Rhamnan Sulphate, heparin, and nitric oxide bind free radicals preventing damages to endothelial cells.

The polysaccharides derived from the seaweed extract, including their sulfated moiety such as Rhamnan Sulphate, heparin, super oxide dismutase and nitric oxide all attack and neutralize free radicals, therefore, diseases associated with cellular injury from free radicals are effectively treated and prevented by the present invention. Also, the polysaccharides derived from the seaweed extract, including their sulfated moiety such as Rhamnan Sulphate aids in the reconstruction of damaged tissue by promoting the production of endogenous heparin, which then forms a complex with and removes extracellular matrix protein accumulations, e.g. fibronectin with consequent reversal or minimization of organ hypertrophy states. Rhamnan Sulphate enhances regeneration of the endothelium following an injury to an endothelium surface.

### EXAMPLE 1

### Extraction Method

Dried green Algae (Monostroma Nitidum) was swollen in 10 Vols. Of water at room temperature for one hour. Thereafter the swollen green algae was ground and refluxed for two (2) hours in a boiling water bath. The water extract was centrifuged (4500 g) for 30 minutes, and the water-soluble polysaccharide in the non-dialyzable fraction was obtained by lyophilization.

The crude polysaccharide was dissolved in water and was applied to a column (2.4×100 cm) of DEAE-cellulose (Whatman DE-52). Starch or neutral polysaccharides were removed by continuous water elution until the sample was completely free as determined by phenol-sulfuric acid detection. Afterwards, acid polysaccharide was fractionated by stepwise alteration of the ionic strength of KCL at 0.5. 0.7 and 2.0 M, and then each fraction was desalted and freeze dried. The 0.5 M KCL fraction (major fraction) successive purification procedures were performed by gel filtration chromatography on a Toyopearl HW-65 (fine) column (1.2×100 cm). The sample was eluted with water at a flow rate of 0.4 ml/min. The major fraction was collected and freeze dried. These procedures or variations of them for extraction of complex polysaccharides are well known.

Composition Analysis of Seaweed Extract

Glycosyl Composition (Polysaccharide Composition)

Glycosyl composition analysis was performed by combined gas chromatography/mass spectrometry (GC/MS) of the per-O-trimethylsilyl (TMS) derivatives of the monosaccharide methyl glycosides produced from the sample by acidic methanolysis.

Methyl glycosides were first prepared from dry sample by methanolysis in 1 M HCl in methanol at 80° C. (18-22 hours), followed by re-N-acetylation with pyridine and acetic anhydride in methanol (for detection of amino sugars). The samples were then per-O-trimethylsilylated by treatment with Tri-Sil (Pierce) at 80° C. (0.5 hours). These procedures were carried out as previously described (Methods Enzymol. 230:1-15; York W. S., Darvill, A. G., McNeil, M., Stevenson, T. T., and Albersheim, P. (1985) Methods Enzymol. 118:340). GC/MS analysis of the TMS methyl glycosides was performed on an HP 5890 GC interfaced to a 5970 MSD, using a All Tech EC-1 fused silica capillary column (30 m×0.25 mm ID).

TABLE 1
Glycosyl Composition Analysis (Commercial Batches)

| Glycosyl residue | Mass (μg) | Mole %¹ |
|---|---|---|
| Rhamnose (Rha) | 657.4 | 69.6 |
| Fucose (Fuc) | n.d. | n.d. |
| Xylose (Xyl) | 57.4 | 6.6 |
| Glucuronic acid (GlcA) | 68.7 | 6.2 |
| Galaturonic acid (GalA) | n.d. | n.d. |
| Mannose (Man) | 20.2 | 2.0 |
| Galactose (Gal) | 33.2 | 3.2 |
| Glucose (Glc) | 128.8 | 12.4 |
| N-acetyl glucosamine (GlcNAc) | n.d. | n.d. |
| 3-deoxy-d-manno-2-octulosonic acid (Kdo) | n.d. | n.d. |
| Arabinose (Ara) | Trace | |
| Rhamnose (Rha) | 888.9 | 63.1 |
| Fucose (Fuc) | n.d. | n.d. |
| Xylose (Xyl) | 102.0 | 7.9 |
| Glucuronic acid (GlcA) | 78.7 | 4.7 |
| Galaturonic acid (GalA) | n.d. | n.d. |
| Mannose (Man) | 47.2 | 3.1 |
| Galactose (Gal) | 68.4 | 4.4 |
| Glucose (Glc) | 260.5 | 16.8 |
| N-acetyl glucosamine (GlcNAc) | n.d. | n.d. |
| 3-deoxy-d-manno-2-octulosonic acid (Kdo) | n.d. | n.d. |
| Arabinose (Ara) | Trace | |
| Rhamnose (Rha) | 907.2 | 70.2 |
| Fucose (Fuc) | n.d. | n.d. |
| Xylose (Xyl) | 80.2 | 6.8 |
| Glucuronic acid (GlcA) | 74.6 | 4.9 |
| Galaturonic acid (GalA) | n.d. | n.d. |
| Mannose (Man) | 29.7 | 2.1 |
| Galactose (Gal) | 41.1 | 2.9 |
| Glucose (Glc) | 186.2 | 13.1 |
| N-acetyl glucosamine (GlcNAc) | n.d. | n.d. |
| 3-deoxy-d-manno-2-octulosonic acid (Kdo) | n.d. | n.d. |
| Arabinose (Ara) | Trace | |
| Rhamnose (Rha) | 1010.9 | 66.8 |
| Fucose (Fuc) | n.d. | n.d. |
| Xylose (Xyl) | 98.2 | 7.1 |
| Glucuronic acid (GlcA) | 97.5 | 5.4 |
| Galaturonic acid (GalA) | n.d. | n.d. |
| Mannose (Man) | 42.7 | 2.6 |
| Galactose (Gal) | 71.1 | 4.3 |
| Glucose (Glc) | 230.2 | 13.8 |
| N-acetyl glucosamine (GlcNAc) | n.d. | n.d. |
| 3-deoxy-d-manno-2-octulosonic acid (Kdo) | n.d. | n.d. |
| Rhamnose (Rha) | 108.8 | 77.3 |
| Xylose (Xyl) | 11.6 | 9.0 |
| Glucuronic acid (GlcA) | 5.5 | 3.3 |
| Mannose (Man) | 2.3 | 1.5 |
| Galactose (Gal) | 5.3 | 3.4 |
| Glucose (Glc) | 10.5 | 5.5 |
| Rhamnose (Rha) | 129.0 | 78.1 |
| Xylose (Xyl) | 12.0 | 8.0 |
| Glucuronic acid (GlcA) | 5.7 | 2.9 |
| Mannose (Man) | 2.9 | 1.6 |
| Galactose (Gal) | 6.1 | 3.4 |
| Glucose (Glc) | 13.2 | 6.0 |
| Rhamnose (Rha) | 216.6 | 74.7 |
| Xylose (Xyl) | 24.5 | 9.2 |
| Glucuronic acid (GlcA) | 13.9 | 4.0 |
| Mannose (Man) | 4.6 | 1.4 |
| Galactose (Gal) | 9.6 | 3.0 |
| Glucose (Glc) | 30.3 | 7.7 |

Bench top analysis of seaweed extracts yielded between approximately 90% to 97% Rhamnose and between approximately 0% and 4.5% Xylose. The seaweed extract contains between approximately 63 mole % to 78 mole % Rhamnose. The seaweed extract contains between approximately 6.5 mole % to 9.2 mole % Xylose. The Rhamnose and Xylose are present in the seaweed extract in amounts between a ratio of approximately 12 Rhamnose to 1 Xylose and approximately 8 Rhamnose to 1 Xylose.

### EXAMPLE 2

### Clinical Studies

Inflammation Risk Biomarkers

An open-label, multiple administration, dose escalation study was conducted to assess the influence of the compound of seaweed extract and L-arginine on vascular function and accepted plasma biochemical markers indicative of endothelial and cardiovascular function.

Method:

Eight healthy subjects (HS), 8 men, aged 39.9±4.0 yrs, eight essential hypertension patients (HT), 5 men and 3 women, aged 57.6±4.4 yrs, and eight peripheral arterial occlusive disease patients (PAOD), 5 men and 3 women, aged 55.6±5.1 years, received three different amounts of proprietary compound (300, 600 and 1200 mg) each over 7 days as an oral dose after 6 hours fasting.

Results and Biochemical Markers:

Selected markers were identified to be relevant to cardiovascular risk assessment; high sensitive C-Reactive Protein (hs-CRP).

Interpretation of these results in human subjects in light of authoritative published studies indicates significant vasoprotective effects from the administration of the proprietary seaweed extract powder and L-arginine compound.

hs-CRP was above the normal range in all hypertensive and all PAOD subjects, and in several healthy subjects. At baseline and at the end of the study (three weeks) the mean concentration of hs-CRP was lower in the healthy group as compared to the hypertensive and PAOD groups.

As is shown in Table 1, all groups demonstrated a decrease in hs-CRP from baseline to the end of the study. At all quartiles of hs-CRP there is a correlation with increased cardiovascular events regardless of LDL cholesterol level.

87% of healthy subjects demonstrated reductions in hs-CRP (up to 43% reduction) during the study. 75% of hypertensive subjects and 62% of PAOD subjects showed reduction of hs-CRP during the study. 80% of all patients in the study with hs-CRP levels greater than 3 times normal reduced their levels during the study. Those with the highest levels showed reductions up to 70% to the baseline values.

The literature suggests that a 30-40% reduction in hs-CRP indicates a reduction in risk of heart attack and stroke risk by at least 30%.

TABLE 2
CRP Test Results
Laboratory test name = CRP, high-sensitiv [mg/dl]

| | n | Mean | Median | SD |
|---|---|---|---|---|
| healthy volunteers | | | | |
| PI - D1 | 8 | 0.127 | 0.069 | 0.145 |
| PI - D7 | 8 | 0.476 | 0.111 | 0.741 |
| PII - D7 | 8 | 0.089 | 0.086 | 0.069 |
| PIII - D7 | 8 | 0.072 | 0.057 | 0.058 |
| hypertension patients | | | | |
| PI - D1 | 8 | 0.463 | 0.204 | 0.673 |
| PI - D7 | 8 | 0.462 | 0.327 | 0.435 |
| PII - D7 | 8 | 0.544 | 0.340 | 0.512 |
| PIII - D7 | 8 | 0.380 | 0.363 | 0.239 |
| PAOD patients | | | | |
| PI - D1 | 8 | 0.454 | 0.248 | 0.538 |
| PI - D7 | 8 | 0.647 | 0.466 | 0.807 |
| PII - D7 | 8 | 0.550 | 0.415 | 0.510 |
| PIII - D7 | 8 | 0.342 | 0.183 | 0.422 |
| Total | | | | |
| PI - D1 | 24 | 0.348 | 0.142 | 0.508 |
| PI - D7 | 24 | 0.528 | 0.295 | 0.656 |
| PII - D7 | 24 | 0.394 | 0.208 | 0.458 |
| PIII - D7 | 24 | 0.265 | 0.142 | 0.304 |

Mechanism of Action:

While not being bound to a mechanism of action, it is thought that the compound of seaweed extract and L-arginine is an endothelial modulating agent due to its direct uptake and preferential binding to endothelial cells.

It is believed that Rhamnan sulfate on the endothelium reduces attachment of inflammatory cells and their passage into healthy tissues. Thus reducing the CRP and IL-6 production, which in turn minimizes the NFKB activation and decreases inflammatory cascade transcription. Since Rhamnan Sulfate reduces this cascade, which is the common pathway to inflammation in cells, it has the activity to retard or prevent inflammation in any cell, tissue type or organ. Rhamnan Sulfate also increases ENOS activity which lowers iNOS activity, thereby limiting the free radical injurious processes in cells and tissues.

Cell surface thrombotic activity directly causes inflammation of the endothelium. The result of these endothelium changes causes increased binding of inflammatory cells from the blood to the endothelium. Upon this attachment, these inflammatory cells induce changes in the endothelium, which permits transmigration through the endothelium into adjacent tissues thereby inducing inflammation in those tissues.

Further, the endothelium in joints susceptible to developing arthritis are treated with Rhamnan Sulfate, there is a decrease in inflammation of the joints.

It is therefore evident how the objective of the present invention is satisfied. First the method and composition of the invention possesses extremely potent anti-thrombotic activity and other inhibitory effects on cell surface coagulation assembly and activity for thrombus inhibition.

Second, since the seaweed extract is from plant cells, it has no potential for the transmission of potentially lethal and serious prion diseases such as mad cow disease.

Third, the seaweed extract has no potential for activating Platelet Factor IV and resulting in immune complex destruction of platelets as seen with heparin administration.

Fourth, the seaweed extract is a functional substitute for heparin in applications requiring systemic (not Plasma) anticoagulant activity such as dialysis, bypass surgery, and polymer tube coatings and devices for use in mammals and humans.

Fifth, the seaweed extract composition has less peptide residues because it is extracted from plant cells as compared to heparin from animal cells. Hence, it is less allergic reaction prone and has fewer immunogenic properties.

Sixth, the seaweed extract of the present invention reduces C-Reactive Protein levels in the plasma.

Seventh, the seaweed extract of the present invention to preserves healthy cardiovascular function including blood vessels, by lowering LDL and/or increasing HDL level in the plasma.

It will be readily apparent to those skilled in the art that many modifications, derivations and improvements are within the scope of the invention. Such modifications, derivations, and improvements should be accorded full scope of protection by the claims appended hereto.

1. A method for retardation of inflammation in a mammal comprising the step of administering to a mammal an amount of seaweed extract in effective doses.

2. The method of claim 1 wherein the seaweed is selected from the group consisting of brown algae, red algae and green algae.

3. The method of claim 1 wherein the seaweed extract contains a polysaccharide.

4. The method of claim 3 wherein the polysaccharide is selected from the group consisting of; Rhamnose, Xylose, Galactose, and Mannose, either individually or a combination thereof.

5. The method of claim 1 wherein the seaweed extract contains between approximately 63 mole % to 78 mole % Rhamnose.

6. The method of claim 1 wherein the seaweed extract contains between approximately 6.5 mole % and 9.2 mole % Xylose.

7. The method of claim 1 wherein Rhamnose and Xylose are present in the seaweed extract in amounts between a ratio of approximately 12 Rhamnose to 1 Xylose and approximately 8 Rhamnose to 1 Xylose.

8. The method of claim 2 wherein the brown algae is selected from the group consisting of Fucus vesiculosus, Fucus Evanescens, Laminaria brasiliensis, or Ascophylum nodosum.

9. The method of claim 2 wherein the green algae is selected from the group consisting of Monostroma nitidium, Monostroma zosteticola, Monostroma angicava, Monostroma lattissimum, Monostroma pulchrum, Monostroma fusem, Monostroma grevillei, Entoromorpha compressa, Ulva arasakii, Ulva Pertussa, Cladophora denna, Cladophora rugulosa, Chaecomorpha spiralis, Chaecomorpha crassa, Spongomorpha duriuscula, Codium fragile, Codium divaricaium Codium latum, or Caulerpa okamarai.

10. The method of claim 1 wherein the dose of seaweed extract is administered to a mammal to treat inflammation related diseases.

11. The method of claim 10 wherein the inflammation related diseases are selected from the group consisting of atherosclerosis, arthritis, colitis, hepatitis, and glial cell and brain cell inflammation.

12. The method of claim 1 further comprising the co-administration of a nitrogen donor together with the seaweed extract.

13. The method of claim 12 wherein the nitrogen donor is selected from the group consisting of L-Arginine and Lysine.

14. The method of claim 1 wherein the seaweed extract is administered orally.

15. The method of claim 1 wherein the seaweed extract is mixed with food stuff; wherein the food stuffs is selected from the group consisting of cereals, bread, drinks, health bars, juices, concentrates, canned food, ice cream, water, staple goods, such as corn, barley, wheat and oat in any form, or taste maskers such as sugar or ascorbic acid.

16. The method of claim 1 wherein a daily dose of seaweed extract is equivalent to between approximately 2,000IU and 200,000IU of heparin activity.

17. The method of claim 1 wherein a daily dose of administered seaweed extract is equivalent to between approximately 5,000IU and 20,000IU of heparin activity.

18. The method of claim 1 wherein a daily dose of seaweed extract is equivalent to between approximately 8,000IU and 12,000IU of heparin activity.

19. The method of claim 1 wherein a daily dose of seaweed extract is approximately 7.5 mg/kg.

20. The method of claim 1 wherein the daily dose of seaweed extract is repeated.

21. A composition for retardation of inflammation in patients comprising: an amount of seaweed extract and a nitrogen donor in effective amounts.

22. The composition of claim 21 wherein the seaweed is selected from the group of brown algae, red algae and green algae.

23. The composition of claim 22 wherein the brown algae is selected from the group consisting of Fucus vesiculosus, Fucus Evanescens Laminaria brasiliensis, or Ascophylum nodosum.

24. The composition of claim 22 wherein the green algae is selected from the group consisting of Monostroma nitidium, Monostroma zosteticola, Monostroma angicava, Monostroma lattissimum, Monostroma pulchrum, Monostroma fusem, Monostroma grevillei, Entoromorpha compressa, Ulva arasakii, Ulva Pertussa, Cladophora denna, Cladophora rugulosa, Chaecomorpha spiralis, Chaecomorpha crassa, Spongomorpha duriuscula, Codium fragile, Codium divaricaium Codium latum, or Caulerpa okamarai.

25. The composition of claim 21 wherein the seaweed extract contains a polysaccharide.

26. The composition of claim 22 wherein the polysaccharide is selected from the group consisting of; Rhamnose, Xylose, Galactose, and Mannose, either individually or a combination thereof.

27. The composition of claim 21 wherein the seaweed extract contains between approximately 63 mole % to 78 mole % Rhamnose.

28. The composition of claim 21 wherein the seaweed extract contains between approximately 6.5 mole % to 9.2 mole % Xylose.

29. The composition of claim 26 wherein Rhamnose and Xylose are present in the seaweed extract in amounts between a ratio of approximately 12 Rhamnose to 1 Xylose and approximately 8 Rhamnose to 1 Xylose.

30. The composition of claim 21 wherein the seaweed extract is mixed with food stuffs.

31. The composition of claim 30 wherein the food stuffs is selected from the group consisting of cereals, bread, drinks, health bars, juices, concentrates, canned food, ice cream, water, staple goods such as corn, wheat, barley, and oat in any form, or taste maskers such as sugar or ascorbic acid.

32. The composition of claim 21 wherein a daily amount of administered seaweed extract is equivalent to between approximately 2,000IU and 200,000IU of heparin activity.

33. The composition of claim 21 wherein a daily amount of administered seaweed extract is equivalent to between approximately 5,000IU and 20,000IU of heparin activity.

34. The composition of claim 21 wherein a daily amount of administered seaweed extract is equivalent to between approximately 8,000IU and 12,000IU of heparin activity.

35. The composition of claim 21 wherein the nitrogen donor is selected from the group consisting of L-Arginine and Lysine.

36. The composition of claim 21 wherein the seaweed is substituted for by its functional analogs, wherein the functional analogs are polysaccharides produced synthetically or derived from plant sources, such as algae, and treat inflammation.

37. A method for preserving a healthy inflammatory response in a mammal comprising the step of administering to a mammal an amount of seaweed extract in effective doses.

38. The method of claim 37 wherein the seaweed is selected from the group of brown algae, red algae and green algae.

39. The method of claim 37 wherein the seaweed extract contains a polysaccharide.

40. The method of claim 39 wherein the polysaccharide is selected from the group consisting of; Rhamnose, Xylose, Galactose, and Mannose, either individually or a combination thereof.

41. The method of claim 37 wherein the seaweed extract contains between approximately 63 mole % to 78 mole % Rhamnose.

42. The method of claim 37 wherein the seaweed extract contains between approximately 6.5 mole % to 9.2 mole % Xylose.

43. The method of claim 37 wherein Rhamnose and Xylose are present in the seaweed extract in amounts between a ratio of approximately 12 Rhamnose to 1 Xylose and approximately 8 Rhamnose to 1 Xylose.

44. The method of claim 38 wherein the brown algae is selected from the group consisting of Fucus vesiculosus, Fucus Evanescens Laminaria brasiliensis, or Ascophylum nodosum.

45. The method of claim 38 wherein the green algae is selected from the group consisting of Monostroma nitidium, Monostroma zosteticola, Monostroma angicava, Monostroma lattissimum, Monostroma pulchrum, Monostroma fusem, Monostroma grevillei, Entoromorpha compressa, Ulva arasakii, Ulva Pertussa, Cladophora denna, Cladophora rugulosa, Chaecomorpha spiralis, Chaecomorpha crassa, Spongomorpha duriuscula, Codium fragile, Codium divaricaium Codium latum, or Caulerpa okamarai.

46. The method of claim 37 wherein preservation of a healthy inflammatory response comprises of reducing the risk of development of artheroscierosis, arthritis, colitis, hepatitis, and glial cell and brain cell inflammation.

47. The method of claim 37 further comprising the co-administration of a nitrogen donor together with the seaweed extract.

48. The method of claim 47 wherein the nitrogen donor is selected from the group consisting of L-Arginine and Lysine.

49. The method of claim 37 wherein the seaweed extract is administered orally.

50. The method of claim 37, wherein the seaweed extract is administered mixed with food stuffs, wherein the food stuffs is selected from the group consisting of cereals, bread, drinks, health bars, juices, concentrates, canned food, ice cream, water, staple goods such as wheat, corn, barley, and oat in any form, or taste maskers such as sugar or ascorbic acid.

51. The method of claim 37 wherein a daily dose of seaweed extract is equivalent to between approximately 2,000IU and 200,000IU of heparin activity.

52. The method of claim 37 wherein a daily dose of seaweed extract is equivalent to between approximately 5,000IU and 20,000IU of heparin activity.

53. The method of claim 37 wherein a daily dose of seaweed extract is equivalent to between approximately 8,000IU and 12,000IU of heparin activity.

54. The method of claim 37 wherein a daily dose of seaweed extract is approximately 7.5 mg/kg.

55. The method of claim 37 wherein the daily dose of seaweed extract is repeated.

56. A composition for preserving a healthy inflammatory response in a mammal comprising; an amount of seaweed extract and a nitrogen donor in effective amounts.

57. The composition of claim 56 wherein the seaweed is selected from the group of brown algae, red algae and green algae.

58. The composition of claim 57 wherein the brown algae is selected from the group consisting of Fucus vesiculosus, Fucus Evanescens, Laminaria brasiliensis, or Ascophylum nodosum.

59. The composition of claim 57 wherein the green algae is selected from the group consisting of Monostroma nitidium, Monostroma zosteticola, Monostroma angicava, Monostroma lattissimum, Monostroma pulchrum, Monostroma fusem, Monostroma grevillei, Entoromorpha compressa, Ulva arasakii, Ulva Pertussa, Cladophora denna, Cladophora rugulosa, Chaecomorpha spiralis, Chaecomorpha crassa, Spongomorpha duriuscula, Codium fragile, Codium divaricaium Codium latum, or Caulerpa okamarai.

60. The composition of claim 56 wherein the seaweed extract contains a polysaccharide.

61. The composition of claim 60 wherein the polysaccharide is selected from the group consisting of; Rhamnose, Xylose, Galactose, and Mannose, either individually or a combination thereof.

62. The composition of claim 56 wherein the seaweed extract contains between approximately 63 mole % to 78 mole % Rhamnose.

63. The composition of claim 56 wherein the seaweed extract contains between approximately 6.5 mole % to 9.2 mole % Xylose.

64. The composition of claim 61 wherein Rhamnose and Xylose are present in the seaweed extract in amounts between a ratio of approximately 12 Rhamnose to 1 Xylose and approximately 8 Rhamnose to 1 Xylose.

65. The composition of claim 56 wherein the seaweed extract is mixed with food stuffs.

66. The composition of claim 65, wherein the food stuffs is selected from the group consisting of cereals, bread, drinks, health bars, juices, concentrates, canned food, ice cream, water, staple goods such as wheat, corn, barley, and oat in any form, or taste maskers such as sugar or ascorbic acid.

67. The composition of claim 56 wherein a daily amount of administered seaweed extract is equivalent to between approximately 2,000IU and 200,000IU of heparin activity.

68. The composition of claim 56 wherein a daily amount of administered seaweed extract is equivalent to between approximately 5,000IU and 20,000IU of heparin activity.

69. The composition of claim 56 wherein a daily amount of administered seaweed extract is equivalent to between approximately 8,000IU and 12,000IU of heparin activity.

70. The composition of claim 56 wherein the nitrogen donor is selected from the group consisting of L-Arginine and Lysine.

71. The composition of claim 56 wherein the seaweed is substituted for by its functional analogs, wherein the functional analogs are polysaccharides produced synthetically or derived from plant sources, such as algae, and treat inflammation.
